(19) United States
(12) Patent Application Publication
(10) Pub. No.: US 2024/0006050 A1
(43) Pub. Date: Jan. 4, 2024

(54) LOCATING AN EPILEPTOGENIC ZONE FOR SURGICAL PLANNING

(71) Applicants: THE JOHNS HOPKINS UNIVERSITY, Baltimore, MD (US); University of Pittsburgh-Of the Commonwealth System of Higher Education, Pittsburgh, PA (US)

(72) Inventors: Sridevi V. SARMA, McLean, VA (US); Kristin M. GUNNARSDOTTIR, Baltimore, MD (US); Jorge A. GONZALEZ-MARTINEZ, Pittsburgh, PA (US)

(73) Assignees: THE JOHNS HOPKINS UNIVERSITY, Baltimore, MD (US); University of Pittsburgh- Of the Commonwealth System of Higher Education, Pittsburgh, PA (US)

(21) Appl. No.: 18/255,727

(22) PCT Filed: Dec. 9, 2021
(86) PCT No.: PCT/US2021/062523

§ 371 Date: Jun. 2, 2023

Related U.S. Application Data
(60) Provisional application No. 63/123,417, filed on Dec. 9, 2020.

(51) Int. Cl. G16H 20/40; G16H 50/20; A61B 5/00; A61B 5/01

(52) U.S. Cl. CPC G16H 20/40; G16H 50/20; A61B 5/7267; A61B 5/4094; A61B 5/015

(57) ABSTRACT

A machine-implemented method, computing device, and at least one non-transitory computer-readable medium are provided. A dynamical network model is parameterized by state transition matrices based on monitored interictal brain data. A node influence-to network score for each respective node is calculated indicating how influential the respective node is. An influenced-by score is calculated for the each respective node indicating an amount by which the respective node is influenced by the nodes. A score is calculated for the each respective node based on a sink index, a source influence index, and a sink connectivity index. Nodes that are in the epileptogenic zone are determined based on the calculated score for each of the nodes. An indication of the nodes in the epileptogenic zone is provided.

This application claims the benefit of U.S. Provisional Patent Application No. 63/123,417 filed in the U.S. Patent and Trademark Office on Dec. 9, 2020, the contents of which are hereby incorporated by reference herein in its entirety.

### STATEMENT OF GOVERNMENTAL INTEREST

This invention was made with government support under grant no. R21NS103113 awarded by the National Institutes of Health. The government has certain rights in the invention.

### BACKGROUND OF THE INVENTION

According to the World Health Organization (WHO), epilepsy is a devastating disease that affects over 50 million people worldwide. About 70% of patients diagnosed with epilepsy respond positively to medication. However, about 30% of patients diagnosed with epilepsy cannot control their seizures with drugs. Interventions include surgically removing or electrically interrupting seizure initiation in an epileptogenic zone (EZ), which is a region or network of regions of a brain from which seizure activity is triggered. Localization and understanding of an anatomical extent of the EZ are important for surgical success. Unfortunately, surgical success rates vary ranging from 30% to 70% with an average of about 50%. Due to the surgical success rates and total expense of diagnostic and surgical treatment (about $200,000 per patient), many surgical candidates decide not to pursue this course of treatment.

Currently, when a patient is diagnosed with epilepsy, a magnetic resonance imaging (MRI) scan and a scalp electroencephalogram (EEG) of a brain of the patient are obtained to determine whether the epilepsy is focal or multi-focal (seizures start in a local region or a few regions before spreading throughout the brain) or generalized (seizures start in several regions simultaneously). Positron emission tomography (PET) scans and single-photon emission computerized tomography (SPECT) scans often are obtained to provide additional evidence of epilepsy type. If seizures are focal and arising from an area in or near a visible lesion on the MRI scan, then patients may go directly to surgery. If seizures are focal but the surgical evaluation is discordant (i.e.: imaging findings are not consistent with scalp ictal (during a seizure) EEG scans, then patients undergo invasive intracranial EEG monitoring to localize the EZ. Noninvasive imaging data and scalp EEGs are used to guide placement of electrodes on or into the brain. Traditionally, placing of the electrodes on or in the brain includes removal of a portion of a skull to gain direct access to the brain, i.e., a craniotomy. An array of electrodes then is placed on a surface of the brain (Electrocorticography, ECoG) from which an intracranial EEG (iEEG) is recorded. Alternatively, depth electrodes, which penetrate into the brain and record iEEG signals not only from a cortical surface but in deep brain structures (stereotactic EEG or SEEG), may be used. SEEG, does not require a craniotomy (removal of a section of the skull), but does require drilling of burr holes into the skull for electrode insertion. Both methods are invasive and require extended stays in a specialized Epilepsy Monitoring Unit (EMU).

Following electrode placement, the patient remains in the EMU for days to weeks waiting for a sufficient number of seizure events, because it is the recordings during these events that are primarily used to predict a location of the EZ. Specifically, epileptologists perform two types of iEEG, interictal (between seizures) and ictal (during seizure). Interictal iEEG data are inspected to identify abnormal “spikes,” also called interictal discharges. Channels on which such spikes are observed are denoted as possible EZ nodes, but it should be noted that spikes have not been proven to be a reliable iEEG marker for the EZ. Ictal (seizure) iEEG data are inspected to identify abnormal activities immediately before seizure onset as well as spread patterns. Seizure events are marked by early presence of beta-band activity (“beta buzz”) or rapid fast intracortical frequencies (>100 Hz), which typically occur milliseconds before the clinical onset of seizures. Assuming the EZ generates abnormal epileptiform activity, which then entrains other regions into a clinical seizure, channels where these onset features first appear are commonly identified as the EZ. Electrodecremental responses (loss of rhythmic activity) are also often observed during seizures. Generally, epileptologists look at a variety of signatures to make their decisions. Because the EEGs are manually interpreted, standard procedure requires collecting several seizure events that are then evaluated by a multidisciplinary clinical team to come to a consensus on which electrodes, or channels, are recording from the EZ.

A final stage of the invasive monitoring involves identifying locations of eloquent cortex (e.g. areas of the brain that control visual, auditory, and motor functions), in order to avoid these areas when planning for surgical removal of the EZ. Determination of the eloquent cortex is done by performing 25-50 Hz periodic cortical stimulation on selected iEEG contacts. In addition, single pulse electrical stimulation (SPES) may be performed to assess effective connectivity of spatially disparate regions that may be involved in seizure onset. Surgery is then planned accordingly if the localized EZ can be sufficiently removed without causing other deficits (e.g. loss in vision, hearing, or motion).

Analyses of iEEG data are subjective and extremely narrow, predominantly relying on signatures occurring right before seizure events. Consequently, identifying interictal or ictal abnormal iEEG events by clinicians amount to analyzing minutes of recordings from more than 8 days of monitoring. Thus, typically less than 1% of the recorded iEEG is actually used for identifying the EZ.

Invasive monitoring is expensive and is associated with a number of complications including, but not limited to, bleeding, infections, and neurological deficits. Costs for a patient stay in an epilepsy medical unit are estimated to be at least $5,000 per day. Even a modest reduction on a length of time required for intracranial monitoring would lead to a large cost saving for hospitals.

Clinicians look for concordance from many disparate and imperfect diagnostic tools such as, for example, MRI, ictal SPECT, scalp EEG and iEEG, but much uncertainty remains with respect to a location of the EZ. Often, a region to be resected is significantly larger than the EZ localized. Resection of this region is irreversible and therefore, is frightening to patients which drives low utility of surgical treatment.

A majority of proposed computational methods of determining an EZ from interictal iEEG data lack consistency in finding objective EEG quantities that correlate to a clinically annotated EZ because the proposed methods fail to capture internal properties of an iEEG network. Proposed algorithms either (i) compute EEG features from individual channels or nodes (e.g. spectral power in a given frequency band), thus ignoring dependencies between channels, or they (ii) apply network-based measures to capture pairwise dependencies in the EEG window of interest. Specifically, correlation or coherence between each pair of EEG channels is computed and organized into an adjacency matrix, on which summary statistics are derived including degree distribution and variants of nodal centrality. Such network-based measures are not based on well formulated hypotheses of the role of the EZ in the iEEG network, and worse, many different networks (adjacency matrices) can have identical summary statistics. The interpretations of such measures are thus ambiguous.

A popular EEG marker that has been proposed and reported in over 1,000 published studies is high-frequency oscillations (HFOs). HFOs are spontaneous events occurring on individual iEEG channels that distinctively stand out from the background signal and are divided into three categories: ripples (80-250 Hz), fast ripples (250-500 Hz), and very-fast ripples (>500 Hz). Regarding epilepsy, retrospective studies suggested that resected brain regions that generate high rates of HFOs may lead to good post-surgical outcome. A 2015 reported meta-analysis investigated whether patients with high HFO-generating areas that had been resected presented a better post-surgical seizure outcome in comparison to patients in whom those areas had not been resected. Although significant effects were found for resected areas that either presented a high number of ripples or fast ripples, effect sizes were small and only a few studies fulfilled their selection criteria. Furthermore, several studies have also questioned the reproducibility and reliability of HFOs as a marker and note that there are also physiologic, non-epileptic HFOs and their existence poses a challenge because disentangling the non-epileptic HFOs from clinically relevant pathologic HFOs still is an unsolved issue. Similar inconclusive results hold in completed prospective studies of HFOs. In 2017, an updated Cochrane review investigated a clinical value of HFOs regarding decision making in epilepsy surgery. Only two prospective studies were identified at the time and the review concluded that there is not enough evidence so far to allow for any reliable conclusions regarding the clinical value of HFOs as a marker for the EZ. Today, five clinical trials are listed as using HFOs for surgical planning on clinicaltrials.gov as either recruiting, enrolling by invitation, or active and not enrolling, and none have reported results.

### SUMMARY OF THE INVENTION

In a first embodiment, a machine-implemented method is provided for identifying an epileptogenic zone for treatment in a brain of a person diagnosed with epilepsy. A dynamical network model is parameterized by state transition matrices based on neural state vectors formed from interictal data generated by monitoring each node of the brain during each consecutive predefined time window. Each of the nodes corresponds to a respective area of the brain being monitored. For each state transition matrix, a corresponding node influence-to-network score and a corresponding node influenced-by-network score are calculated for each node. The corresponding node influence-to-network score indicates how influential the respective node is regarding the each of the plurality of nodes, and the node influenced-by score indicates an amount by which the respective node is influenced by the remaining network nodes. For the each state transition matrix, a sink index, a source influence index, and a sink connectivity index are calculated for the each respective node. The sink index for each respective node indicates how far the each respective node is from an ideal sink when one of rows and columns of a two-dimensional representation of the nodes are arranged according to a rank of the each respective node with respect to the node influence-to network score and another of the rows and the columns of the two-dimensional representation of the nodes is arranged according to a rank of the each respective node with respect to the node influenced-by network score. The source influence index for each respective node is then calculated based on a sum of an influence of all of the nodes on a respective node weighted by a source index of each node. The sink connectivity index of the each respective node is based on a sum of an influence of all of the nodes on the respective node weighted by a sink index of the each node. A score for each respective node is calculated based on the source influence index, the sink index, and the sink connectivity index for the respective node. The nodes that are in the epileptogenic zone are determined based on the calculated score for each of the nodes over all of the state transition matrices. An indication of the nodes determined to be in the epileptogenic zone is provided for clinicians to plan a surgical treatment involving the epileptogenic zone.

In a second embodiment, a computing device for aiding a clinician to diagnose a patient as having epilepsy is provided. The computing device includes at least one processor and a memory connected to the at least one processor. The at least one processor is configured to perform a method. According to the method, a dynamical network model is parameterized by state transition matrices based on neural state vectors formed from interictal data generated by non-invasively monitoring each node of a brain during consecutive predefined time windows. Each of the nodes corresponds to a respective area of the brain being monitored. For each of the state transition matrices, a node influence-to network score and a node influenced-by network score, respectively, are calculated for each respective node. The node influence-to network score indicates how influential the respective node is regarding the each of the nodes. The node influenced-by network score indicates an amount by which a respective node is influenced by the nodes. For each respective state transition matrix corresponding to a respective predefined time window, a respective score for the each respective node is calculated as a function of, based on the respective state transition matrix, a sink index for the respective node, a source influence index for the respective node, and a sink connectivity index for the respective node to produce the respective score for the each respective node for the respective predefined time window. The sink index for the each respective node indicates how far each respective node is from an ideal sink when one of rows and columns of a two-dimensional representation of the nodes is arranged according to a rank of the each respective node with respect to the node influence-to network score and another of the rows and the columns of the two-dimensional representation of the nodes is arranged according to a rank of the each respective node with respect to the node influenced-by network score. The source influence index for the each respective node is based on a sum of an influence of all of the nodes on a respective node weighted by a source index of the respective node. The sink connectivity index for the each respective node is based on a sum of an influence of all of the nodes on a respective node weighted by the sink index of the respective node. A mean score for each of nodes is calculated based on the calculated score for each of the nodes over the each respective state transition matrix. The mean score for the each of the plurality of nodes is normalized. A number of nodes having mean scores greater than

$$\frac{1}{N} ,$$

where N is a total number of nodes, is counted. When the count of the number of nodes is greater than a predefined percentage of the total number of nodes, epilepsy is indicated. When the count of the number of nodes is less than or equal to the predefined percentage of the total number of nodes, a healthy brain is indicated.

In a third embodiment, at least one non-transitory computer-readable storage medium having computer instructions stored thereon for identifying an epileptogenic zone in a brain of a person diagnosed with epilepsy is provided. When executed by at least one processor of a computing device, the computing device is configured to perform a method. According to the method, a dynamical network model is parameterized by state transition matrices based on neural state vectors formed from interictal data generated by invasive monitoring of each node of the brain during each consecutive predefined time window. Each of the nodes corresponds to a respective probe implanted in a respective area of the brain. A sink index, a source influence index, and a sink connectivity index are calculated for the each of the nodes. The sink index for the each respective node indicates how far the each respective node is from an ideal sink when one of rows and columns of a two-dimensional representation of the nodes is arranged according to a rank of the each respective node with respect to the node influence-to network score and another of the rows and the columns of the two-dimensional representation of the nodes is arranged according to a rank of the each respective node with respect to the node influenced-by network score. The source influence index for each respective node is based on a sum of an influence of the all of the nodes on a respective node weighted by a source index of each node. The sink connectivity index of the each respective node is based on a sum of an influence of the plurality of nodes on the each respective node weighted by a sink index of each node. A score for the each respective node is calculated as a function of an average of the sink index, an average of the source influence index, and an average of the sink connectivity index for the respective node over the state transition matrices. Nodes that are in the epileptogenic zone are determined based on the calculated score for the each respective node. An indication of the determined nodes in the epileptogenic zone are provided for clinicians to plan a surgical treatment involving the epileptogenic zone.

### BRIEF DESCRIPTION OF THE DRAWINGS

FIG. 1 illustrates an example environment in which various embodiments may operate.

FIG. 2 is an example diagram of a processing device that may implement a server or a computing device in the example environment shown by FIG. 1.

FIG. 3 illustrates an example workflow that includes monitoring nodes (i.e. channels of electrodes) placed in different areas of a brain of a patient to generate interictal iEEG data that includes multiple neural state vectors (one neural state vector per node for each respective predefined time window), from which dynamical network models (DNMs) may be derived.

FIG. 4 shows an example of 4 nodes in an N node iEEG network, one of which is a source node that influences other nodes and another of which is determined to be a sink node that is highly influenced by the source node.

FIG. 5 shows an example two-dimensional representation of the nodes in an N node iEEG network in which columns are ranked according to an influence of nodes on each respective node and rows are ranked according to an influence of nodes on respective nodes.

FIG. 6 is a flowchart of an example process for determining nodes that are in an epileptogenic zone based on influential relationships among the nodes.

FIG. 7 is a flowchart of an example process for training a model to estimate a probability of a successful surgical outcome based on influential relationships among the nodes.

FIG. 8 shows a flowchart of an example process for generating a heat map indicating, by colors, ranges of scores for respective nodes based on influential relationships among nodes in an iEEG network over multiple consecutive time windows.

### DETAILED DESCRIPTION OF THE INVENTION

### Definition of Terms

Node Influence-To-Network Score: A node influence-to-network score may be calculated for each of the nodes in a state transition matrix. For example, in embodiments in which each column of a state transition matrix, A, has values representing an influence of a respective node in an interictal intracranial EEG (iEEG) network on the remaining network nodes, and each row of the A matrix has values representing an amount of which a respective node is influenced by the remaining nodes in the iEEG network, the node influence-to-network score of node i can be calculated for each state transition matrix of an N-node network according to Σⱼ₌₁^{N} Aⱼᵢ.

Node Influenced-By-Network Score: A node influenced-by-network score may be calculated for each of the nodes in a state transition matrix. For example, in embodiments in which each column of a state transition matrix, A, has values representing an influence of a respective node on each remaining node in an interictal intracranial EEG (iEEG) network, and each row of the A matrix has values representing an amount of which a respective node is influenced by the remaining nodes in the iEEG network, the node influenced-by network score of node i can be calculated for each state transition matrix of an N-node network according to Σⱼ₌₁^{N} Aⱼᵢ.

Sink Index: A sink index for each respective node indicates how far the each respective node is from an ideal sink when one of rows and columns of a two-dimensional representation of the nodes is arranged according to a rank of the each respective node with respect to a node influence-to-network score and another of the rows and columns of the two-dimensional representation of the nodes is arranged according to a rank of the each respective node with respect to the node influence-by-network score. The sink index of node, or channel, i may be calculated according to

$${sink}_{i} = \sqrt{2} - ‖ ( {r}_{i} , {c}_{i} ) - ( 1 , \frac{1}{N} ) ‖$$

where (rᵢ, cᵢ) corresponds to a row and column rank of a node, or channel, i. A value of the row rank and the column rank for an ideal sink is

$$( 1 , \frac{1}{N} ) .$$

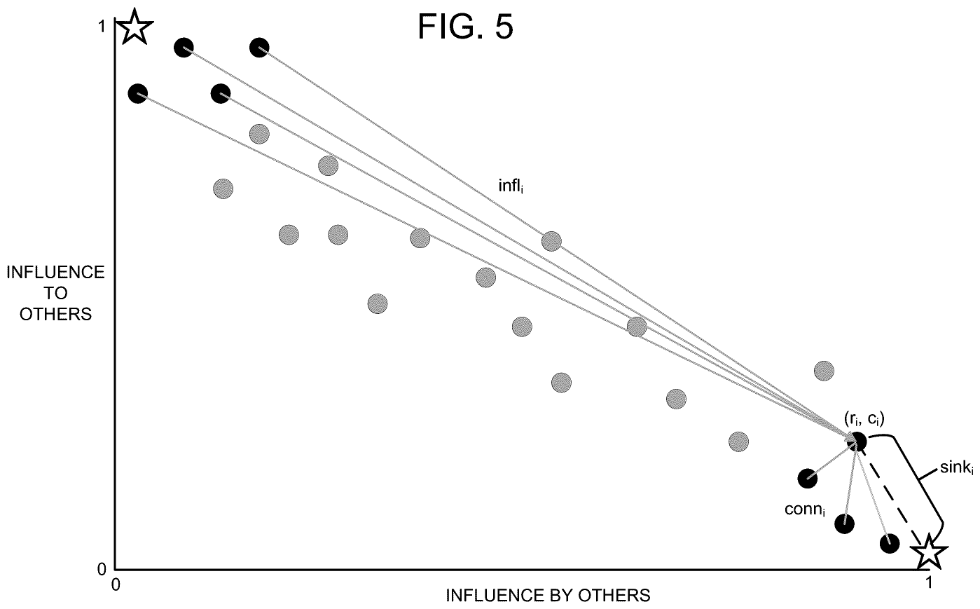

### See FIG. 5.

Source Index: a source index for each respective node indicates how far the each respective node is from an ideal source when one of rows and columns of a two-dimensional representation of the nodes is arranged according to a rank of the each respective node with respect to the node influence-to-network score and another of the rows and columns of the two-dimensional representation of the plurality of nodes is arranged according to a rank of the each respective node with respect to the node influenced-by-network score. The source index may be calculated according to

$${source}_{i} = \sqrt{2} - ‖ ( {r}_{i} , {c}_{i} ) - ( \frac{1}{N} , 1 ) ‖$$

where the ideal source index has a row and column rank of

$$( \frac{1}{N} , 1 ) .$$

### See FIG. 5.

Source Influence Index: a source influence index quantifies how much sources influence a node, or channel, i. The source influence index may be calculated according to

inflᵢ^{w}=Σⱼ₌₁^{N} abs(Aᵢⱼ)×sourceⱼ^{w}

where w is a time window. A high source influence suggests that node, or channel, i received strong influences from sources in the interictal dynamical network model (DNM).

Sink Connectivity Index: A sink connectivity index quantifies a strength of connections from top sinks to node, or channel, i. The sink connectivity index may be calculated according to

connᵢ^{w}=Σⱼ₌₁^{N} abs(Aᵢⱼ)×sinkⱼ^{w}

where w refers to a time window.

### DESCRIPTION OF EMBODIMENTS

In various embodiments, a predictive power of dynamical network models (DNMs) leverage iEEG data collected to assist in localizing an EZ. DNMs are generative models that capture how every channel or node interacts with every other channel or node dynamically. A DNM based on interictal iEEG data takes the form of a linear time-varying (LTV) DNM that mathematically describes how each observed region of a brain, i.e., an iEEG contact signal or node, interacts with other regions during spontaneous neural activity. The LTV DNM may be constructed by concatenating a sequence of linear time-invariant (LTI) DNMs derived in predefined equal consecutive time windows of the iEEG data. In one embodiment, the predefined equal consecutive time windows are 500 millisecond windows in a form of:

x(t+1)=Ax(t)

where x(t)∈ [symbol] ^{n×1} is denoted as a neural state vector, A∈ [symbol] ^{n×n} is a state transition matrix, [symbol] and n is a number of channels or nodes.

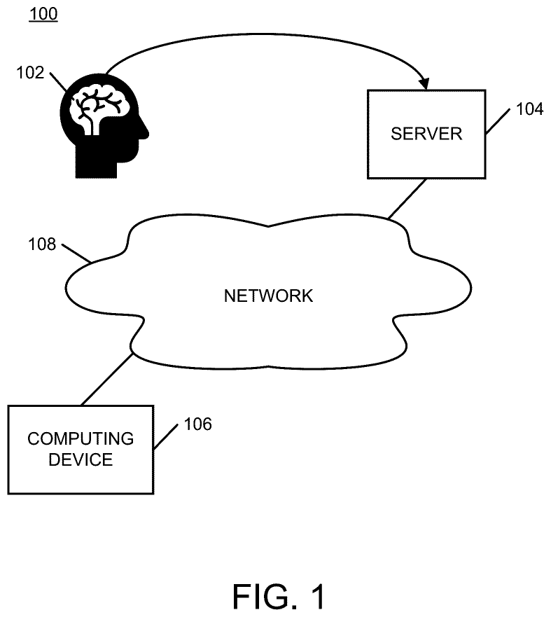

FIG. 1 shows an example environment 100 in which various embodiments may operate. Invasive or non-invasive monitoring of interictal data of a brain of a patient 102 diagnosed with epilepsy may be recorded to a server 104. The monitoring may be performed via iEEG, or any other method of recording electrical signals or magnetic fields in the brain. Server 104 may include a single server or multiple servers configured as a server farm. A computing device 106 may receive the recorded interictal data from server 104 via a network 108 and may analyze the interictal data to determine nodes that are sources and other nodes that are sinks, as well as nodes that are determined to be in an EZ. Network 108 may include a direct wired or wireless connection between server 104 and computing device 106 in some embodiments. In other embodiments, network 108 may include a single network or a network of networks such as, for example, a packet switching network, a local area network, a wide area network, an Internet, as well as other types of networks. Server 104 and computing device 106 may have a wired or wireless connection to network 108.

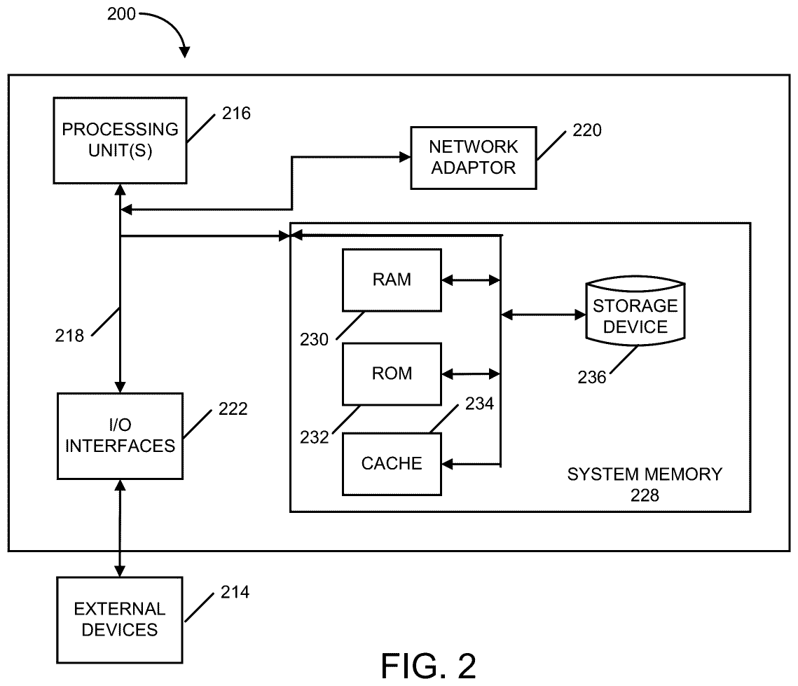

FIG. 2 illustrates an example computing system 200 that may implement any of server 104 and/or computing device 106. Computing system 200 is shown in a form of a general-purpose computing device. Components of computing system 200 may include, but are not limited to, one or more processing units 216, a system memory 228, and a bus 218 that couples various system components including system memory 228 to one or more processing units 216.

Bus 218 represents any one or more of several bus structure types, including a memory bus or memory controller, a peripheral bus, an accelerated graphics port, and a processor or local bus using any of a variety of bus architectures. Such architectures may include, but not be limited to, Industry Standard Architecture (ISA) bus, Micro Channel Architecture (MCA) bus, Enhanced ISA (EISA) bus, Video Electronics Standards Association (VESA) local bus, and Peripheral Component Interconnects (PCI) bus.

Computing system 200 may include various non-transitory computer system readable media, which may be any available non-transitory media accessible by computing system 200. The computer system readable media may include volatile and non-volatile non-transitory media as well as removable and non-removable non-transitory media.

System memory 228 may include non-transitory volatile memory, such as random access memory (RAM) 230 and cache memory 234. System memory 228 also may include non-transitory non-volatile memory including, but not limited to, read-only memory (ROM) 232 and storage system 236. Storage system 236 may be provided for reading from and writing to a non-removable, non-volatile magnetic medium, which may include a hard drive or a Secure Digital (SD) card. In addition, a magnetic disk drive, not shown, may be provided for reading from and writing to a removable, non-volatile magnetic disk such as, for example, a floppy disk, and an optical disk drive for reading from or writing to a removable non-volatile optical disk such as a CD-ROM, DVD-ROM or other optical media. Each memory device may be connected to bus 218 by at least one data media interface. System memory 228 further may include instructions for processing unit(s) 216 to configure computing system 200 to perform functions of embodiments of the invention. For example, system memory 228 also may include, but not be limited to, processor instructions for an operating system, at least one application program, other program modules, program data, and an implementation of a networking environment.

Computing system 200 may communicate with one or more external devices 214 including, but not limited to, one or more displays, a keyboard, a pointing device, a speaker, at least one device that enables a user to interact with computing system 200, and any devices including, but not limited to, a network card, a modem, etc. that enable computing system 200 to communicate with one or more other computing devices. The communication can occur via Input/Output (I/O) interfaces 222. Computing system 200 can communicate with one or more networks including, but not limited to, a local area network (LAN), a general wide area network (WAN), a packet-switched data network (PSDN) and/or a public network such as, for example, the Internet, via network adapter 220. As depicted, network adapter 220 communicates with the other components of computer system 200 via bus 218.

It should be understood that, although not shown, other hardware and/or software components could be used in conjunction with computer system 200. Examples, include, but are not limited to, microcode, device drivers, redundant processing units, external disk drive arrays, RAID systems, tape drives, and data archival storage systems, etc.

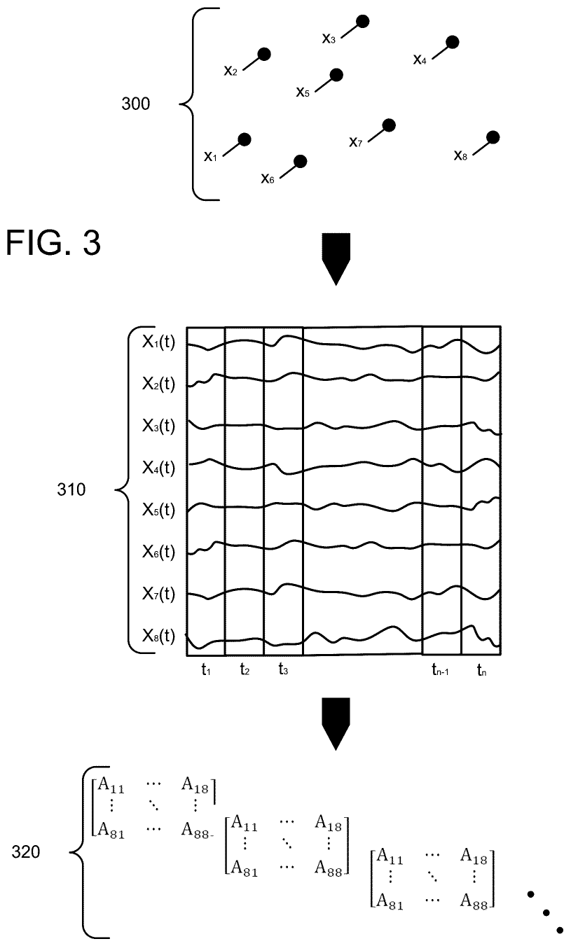

FIG. 3 shows an example workflow of various embodiments. The workflow includes a computing device receiving recorded results of monitoring nodes (i.e. channel contacts) 300, which include multiple neural state vectors 310, shown in FIG. 3 as rows of values for each node (nodes x₁ to x₈ in an 8-node network) during a sequence of n multiple equal consecutive predetermined time windows, tᵢ to tₙ. The equal consecutive predetermined time windows may each be 500 milliseconds, one second, or another suitable length of time. The neural state vector for each of the nodes in each equal predetermined time window may be parameterized as multiple state transition matrices shown in a form of respective A matrices 320, which estimate a DNM. The DNM is generative and can simulate iEEG time-series data over time.

In at least some embodiments, each column of an A matrix has values representing an influence of each respective node in the iEEG network on the remaining nodes, and each row of the A matrix has values representing an amount by which each respective node is influenced by the remaining nodes in the iEEG network. For example, with respect to an 8-node network with reference to A matrices 320, A₁₁ is a value representing an influence of node x₁ on itself, A₁₂ is a value representing an influence of node x₂ on node x₁, . . . , and node A₁₈ is a value representing an influence of node x₈ on node x₁. With respect to columns of an A matrix, A₁₈ is a value representing how much node x₁ is influenced by node x₈, A₂₈ is a value representing how much node x₂ is influenced by node x₈ . . . , and A₈₈ is a value representing how much node x₈ is influenced by itself.

In other embodiments, each column of an A matrix has values representing an influence of a respective node on each node in the iEEG network, and each row of the A matrix has values representing an influence of each node in the iEEG network on a respective node.

In the various embodiments, an LTV DNM parameterized from recorded interictal iEEG may be used to identify two groups of nodes in the iEEG network. The two groups of nodes are nodes that are continuously inhibiting neighboring nodes (denoted as source nodes) and the nodes that are being inhibited by the source nodes (denoted as sink nodes).

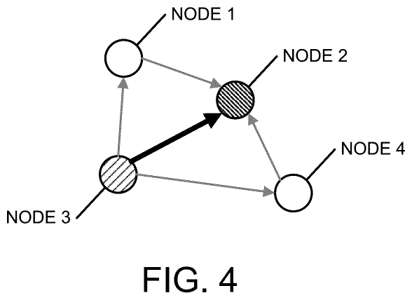

FIG. 4 shows four nodes of an n-node network. Node 3 is a source node because it is highly influential on all nodes, specifically node 2. On the other hand, node 2 is a sink node because it is highly influenced by other nodes, including node 3 in the network, and does not necessarily influence any other node.

FIG. 5 is a two-dimensional representation of nodes in which a position of each respective node aligned with a vertical axis indicates a ranking of the respective node regarding its node influence-to-network score, and a position of each respective node aligned with a horizontal axis indicates a ranking of the respective node regarding the node influence-by-network score. Thus, a first star in an upper left portion of FIG. 5 indicates a position of an ideal source node, a source node having a high ranking with respect to its influence on other nodes and a low ranking with respect to being influenced by the other nodes, and a second star in a lower right portion FIG. 5 indicates a position of an ideal sink node, a sink node having a high ranking with respect being influenced by other nodes and a low ranking with respect to influencing other nodes. In this example two-dimensional representation, source nodes tend to be positioned in an upper left portion and sink nodes tend to be positioned in a lower right portion of the two-dimensional representation. As shown in FIG. 5, an ideal sink node has a lowest ranking with respect to its influence on other nodes and has a highest ranking with respect to being influenced by other nodes. An ideal source node has a highest ranking with respect to its influence to other nodes and has a lowest ranking with respect to being influenced by other nodes.

In other embodiments, a position of each respective node aligned with a vertical axis indicates a ranking of the respective node regarding being influenced by other nodes, and a position of each respective node aligned with a horizontal axis indicates a ranking of the respective node regarding its influence on the other nodes.

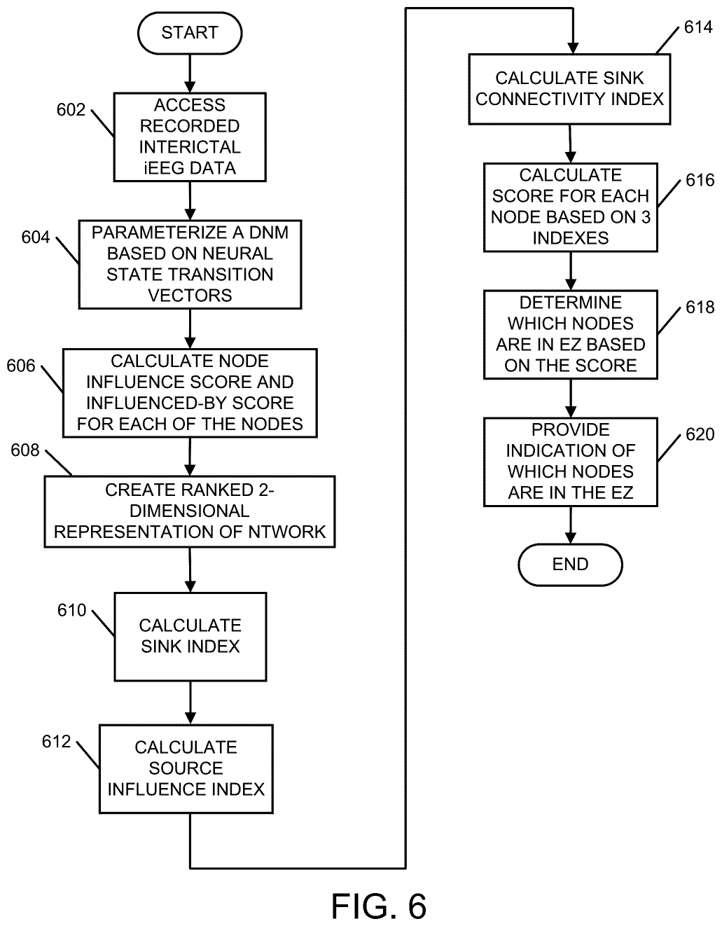

FIG. 6 is a flowchart of a process that may be executed by computing device 106 in various embodiments. The process may begin by accessing or obtaining interictal iEEG data recorded by multiple contacts or nodes implanted in a brain of a patient (act 602). For each consecutive equal time window, an LTV DNM may be parameterized from neural state transition vectors derived from the iEEG data for each node during each consecutive equal time window (act 604), thereby forming A matrices (i.e. state transition matrices).

A node influence-to network score and a node influenced-by-network score may be calculated for each of the nodes in each state transition matrix (act 606). For example, in embodiments in which each column of a state transition matrix, A, has values representing an influence of a respective node in the iEEG network on the remaining nodes, and each row of the A matrix has values representing an amount by which a respective node is influenced by the remaining nodes in the iEEG network, the node influence-to network score of node i can be calculated for each state transition matrix of an N-node network according to Σⱼ₌₁^{N} Aⱼᵢ and the node influenced-by network score of node i can be calculated for each state transition matrix according to Σⱼ₌₁^{N} Aᵢⱼ.

Next, a two-dimensional representation of the nodes, as previously discussed with respect to FIG. 5, may be created for the each respective state transition matrix (act 608) and a sink index may be calculated for each of the nodes with respect to the each respective state transition matrix (act 610). The sink index may capture how close a node is to a position of an ideal sink node on the two-dimensional representation of the nodes. The larger the sink index is for a node, the more likely the node is a sink node. In various embodiments, the sink index of node i for the each respective state transition matrix may be calculated according to,

$${sink}_{i} = \sqrt{2} - ‖ ( {r}_{i} , {c}_{i} ) - ( 1 , \frac{1}{N} ) ‖$$

where sinkᵢ represents the sink index of node i, (rᵢ, cᵢ) is a row and column position of node i in the two-dimensional representation, and

$$( 1 , \frac{1}{N} )$$

is a row and column position of an ideal sink node in the two-dimensional representation of an N-node network. The sink index is equivalent to a Euclidean distance of a position of node i from an ideal sink node in the two-dimensional representation, subtracted from the maximum possible distance from the ideal sink in the two-dimensional representation.

Similar to the sink index, the source index captures how close a channel is to the ideal source

$$( r = \frac{1}{N} and c = 1 ) .$$

The source index may be calculated as:

$${source}_{i} = \sqrt{2} - ‖ ( {r}_{i} , {c r}_{i} ) - ( \frac{1}{N} , 1 ) ‖ \qquad ( 1 )$$

The larger the source index, the more likely channel i is a source.

Next, a source influence index may be calculated for each of the nodes for the each respective state transition matrix (act 612). The source influence index captures a sum of an influence of all nodes in a network on a respective node weighted by a source index of each node. The higher a value of the source influence index is for a respective node, the more influence source nodes have on the respective node. For example, the source influence index of node i for a respective state transition matrix may be calculated according to inflᵢ^{w}=Σⱼ₌₁^{N} abs (Aᵢⱼ)*sourceⱼ^{w}, where sourceᵢ is the source influence index of node i.

A sink connectivity index may be calculated for each of the nodes for the each respective state transition matrix (act 614). The sink connectivity index of node i for a respective state transition matrix of a time window, w, may be calculated according to

connᵢ^{w}=Σⱼ₌₁^{N} abs(Aᵢⱼ)*sinkⱼ^{w}

where connᵢ represents the sink connectivity index of node i in time window w and sinkⱼ^{w} represents the sink index of node j in time window w.

Scores for each of the nodes may be calculated based on an average value of the each respective node's source influence index, sink connectivity index, and sink index over the state transition matrices (act 616). In some embodiments, a source-sink index score for node i may be calculated as a function (e.g., the product) of node i's average source influence index, average sink connectivity index, and average sink index. Which of the nodes are included in an EZ then may be determined based on the scores of the nodes (act 618). For example, nodes having a score greater than a high score threshold value may be determined to be located in the EZ. In some embodiments, the high score threshold value may be set such that nodes having a score greater than a top predefined percentage may be determined to be located in the EZ. In various embodiments, the high score threshold value may be set to, for example, a top 5%, a top 10%, a percentage value between 5% and 10%, or another percentage.

Computing device 106 may then provide an indication of which nodes are located in the EZ (act 620). The indication may be presented on a display screen, may be printed in a report, may be announced in a computer generated voice over a speaker, or may be provided in some other manner.

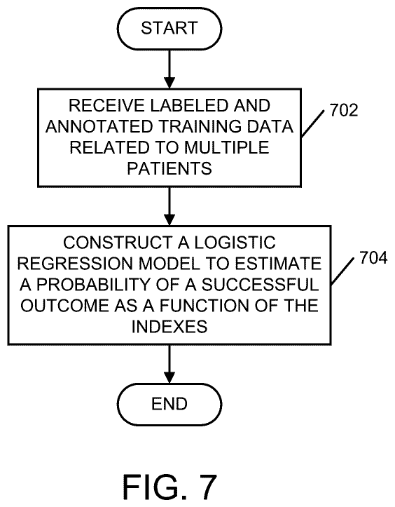

In various embodiments, a model may be trained to predict a probability of a successful outcome, pₛ. FIG. 7 is a flowchart illustrating an example process for training and using such a model to predict a probability of a successful outcome. The process may begin with a computing device such as, for example, computing device 106, receiving labeled and annotated training data related to multiple patients (act 702). The training data may include clinically annotated and/or labeled iEEG data for each of the patients. For each of the patients, nodes may be labeled as being in a seizure onset zone (SOZ), an early propagation zone (EPZ), or as other. Nodes labeled as being in the SOZ demonstrate earliest electrophysiological changes during an ictal (seizure) event and generally precede a clinical onset of seizures. Nodes labeled as being in the EPZ are involved at a time of earliest clinical (semiological) manifestations during an ictal event. A clinically annotated EZ is an anatomical area to be treated (resected or ablated) in order to permanently extinguish epileptiform activity. The clinically annotated EZ is defined as a combination of the SOZ and the EPZ. Successful outcomes are defined as being seizure free or nearly seizure free at more than 12 months post-op.

A predictive model (e.g., a logistic regression model) may be constructed to estimate a probability of a successful surgical outcome, pₛ (act 704). In some embodiments, pₛ may be estimated as a function of the sink index and the source influence index as follows based on a training data set for a number of patients:

$$log ( \frac{{p}_{s}}{1 - {p}_{s}} ) = {β}_{0} + {β}_{1} ( {sink}_{E Z} - {sink}_{n o n E Z} ) + {β}_{2} ( s r {c}_{E Z} - s r {c}_{n o n E Z} ) ,$$

where pₛ is a probability of a successful outcome, β₀, β₁ and β₂ are constants, sink_{EZ} is an average sink index over all nodes located in the clinically annotated EZ, sink_{nonEZ} is the average sink index over all nodes located outside of the clinically annotated EZ, src_{EZ} is an average source influence index over all of the nodes located in the clinically annotated EZ, and src_{nonEZ} is an average source influence index for all of the nodes located outside of the clinically annotated EZ. The above logistic regression model may be solved by determining a maximum likelihood estimation.

In some other embodiments, the predictive model (e.g., logistic regression model) may be constructed to estimate a probability of a successful surgical outcome, pₛ, as a function of the sink index, the source influence index, and the sink connectivity index as

follows based on a training data set for a number of patients according to: log

$$log ( \frac{{p}_{s}}{1 - {p}_{s}} ) = {β}_{0} + {β}_{1} ( {sink}_{E Z} - {sink}_{n o n E Z} ) + {β}_{2} ( s r {c}_{E Z} - s r {c}_{n o n E Z} ) + {β}_{3} ( c o n {n}_{E Z} - c o n {n}_{n o n E Z} ) ,$$

where β₃ is
a constant, conn_{EZ} is an average sink connectivity index for all of the nodes located in the clinically annotated EZ, and conn_{nonEZ} is an average sink connectivity index for all of the nodes located outside of the clinically annotated EZ.

After training the model, the probability of success, pₛ, may be estimated from iEEG data according to either of the abovementioned logistic regression models, where sink_{EZ} is an average sink index of all nodes determined to be in the EZ, sink_{nonEZ} is an average sink index of all of the nodes determined to be outside of the EZ, src_{EZ} is an average source influence index for all of the nodes determined to be in the EZ, src_{nonEZ} is an average source influence index for all of the nodes determined to be outside of the EZ, conn_{EZ} is an average sink connectivity index for all of the nodes determined to be in the EZ, and conn_{nonEZ} is an average sink connectivity index for all of the nodes determined to be outside of the EZ.

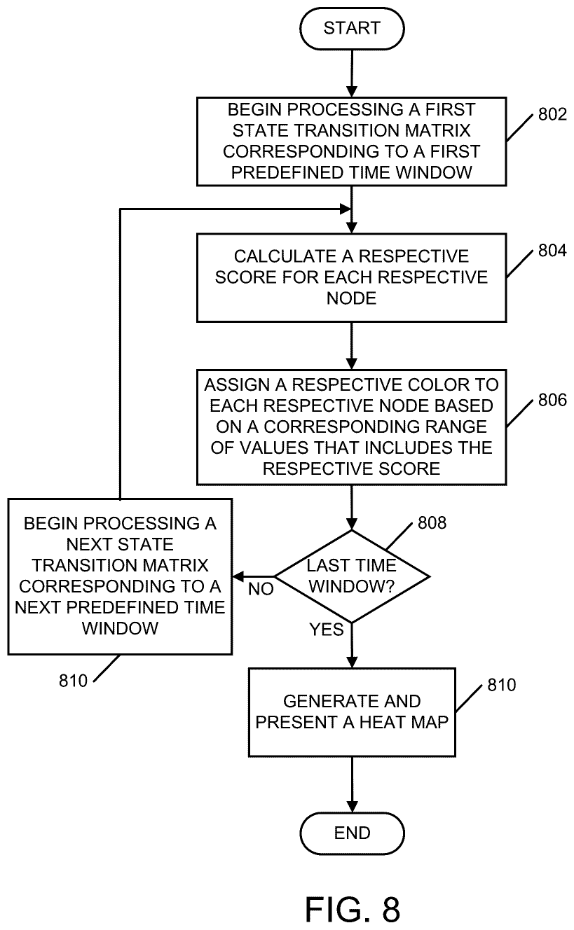

In some embodiments, a heat map may be generated and presented. The heat map may represent each node as a respective row and each predetermined consecutive time window as a column. A cell at an intersection of a row and a column, corresponds to a particular node (based on the row) at a particular time window (based on the column). As previously mentioned, a score may be calculated for each node in each respective time period. A color may be assigned to each cell based on a particular range of scores which includes a score for a corresponding node and time window. FIG. 8 is a flowchart of an example process that may execute on computing device 106 for generating and presenting a heat map.

The process of FIG. 8 may begin by processing a first state transition matrix corresponding to a first predefined time window (act 802). A respective score may be calculated for each respective node of the respective time window (act 804). For example, a node influence-to score and an influenced-by score may be calculated for each of the nodes based on a state transition matrix corresponding to the respective time window. Based on the node influence-to network and node influenced-by network scores, a sink index score, a source influence index score, and a sink connectivity index score may be calculated for each respective node. In some embodiments, a source-sink index score may be calculated as a function (e.g., by multiplying) the sink index, the source influence index and the sink connectivity index. A color corresponding to a respective time window score may be assigned to each cell based on a corresponding range of scores in which each score is included (806).

After all nodes are assigned colors for a time window, a determination may be made regarding whether the last time window was processed (act 808). If the last time window was not processed, then acts 804-808 again may be performed to calculate time window scores in a next time window and assign colors corresponding to the time window scores.

If, during act 808, the last time window was processed, then a heat map is generated and presented (act 810). The heat map may be presented in any of a number of ways including, but not limited to, presented on a display screen, printed, generated as a file, and sent as an attachment to an email.

Embodiments may determine whether a patient has epilepsy based on non-invasive monitoring of each node of a brain of the patient during each consecutive predefined time window to produce interictal data. Each of the nodes corresponds to a respective area of the brain being monitored. The noninvasive monitoring may include, but not be limited to, a scalp EEG, functional magnetic resonance imaging (FMRI), a magnetoencephalogram, or other non-invasive methods. A DNM may be parameterized by multiple state transition matrices based on multiple neural state vectors formed from the interictal data generated by the noninvasive monitoring during each consecutive predefined time window. As previously described, the computing device may calculate for each respective state transition matrix, a node influence-to network score for each respective node indicating how influential the respective node is regarding each of the nodes in an interictal network. Based on the each respective state transition matrix, the computing device may calculate a node influenced-by network score for the each respective node indicating an amount by which the respective node is influenced by the nodes of the interictal network during a corresponding time window.

The computing device may calculate a respective mean score for the each respective node based on the multiple state transition matrices. The respective mean score may be calculated as a function (e.g., by multiplying), based on the multiple state transition matrices, an average source influence index for the respective node, an average sink index for the respective node, and an average sink connectivity index for the respective node to produce the respective scores for the each respective node. The computing device then may normalize all mean scores such that a sum of all mean scores is equal to one. Next, the computing device may count a number of nodes having mean scores greater than

$$\frac{1}{N} ,$$

where N is a total number of nodes. If the count is greater than a predefined percentage (e.g., 30%) of N, then a diagnosis of epilepsy is indicated. A healthy brain is indicated when the count is less than or equal to the predefined percentage of N. The computing device may present an indication of whether the brain is diagnosed as an epileptic brain or a healthy brain. The indication may be displayed on a display screen, printed in a report, sent via an email, or may be provided via another method.

In the embodiments that generate and display a heat map, a distribution of colors in the heat map may be indicative of whether the brain is a healthy brain or an epileptic brain.

An average surgical success rate is approximately 50% via standard of care. Prediction based on a probability of a successful surgical outcome, pₛ, was accurate at about 73±4.7% of the time when applied to a dataset of 65 patients (28 successes and 37 failures). Typically, successful outcomes had top source nodes pointing to top sink nodes, and sink nodes had high connectivity. In contrast, failed outcomes had top source nodes pointing to both top sink nodes and other non-sink nodes, and the sink nodes had high connectivity with each other as well as with the other non-sink nodes that the top source nodes influence.

Various embodiments provided a number of advantages over standard of care methods. The advantages include a much shorter length of time for invasive intracranial monitoring of activity in different areas of a brain, thereby reducing a risk of infection and decreasing a length of a hospital stay. Further, by monitoring brain activity between seizures, more consistent objective results are provided. In addition, more precise and more focal and limited surgical resections are provided by new information from what currently was largely ignored iEEG data. Finally, the various embodiments enable caregivers to better interpret their EEG recordings.

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising”, “includes”, “including”, “has”, “have”, “having”, “with” and the like, when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description but is not intended to be exhaustive or limited to the invention in the form disclosed. Many modifications and variations will be apparent to those of ordinary skill in the art without departing from the scope and spirit of the invention. The embodiments were chosen and described in order to best explain the principles of the invention and the practical application, and to enable others of ordinary skill in the art to understand the invention for various embodiments with various modifications as are suited to the particular use contemplated.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or improvement over conventional technologies, or to enable others of ordinary skill in the art to understand the embodiments disclosed herein.

Aspects of the present invention are described herein with reference to flowchart illustrations and/or block diagrams of methods, apparatus (systems), and computer-readable storage devices having instructions stored therein for carrying out functions according to embodiments of the invention. It will be understood that each block of the flowchart illustrations and/or block diagrams, and combinations of blocks in the flowchart illustrations and/or block diagrams, can be implemented by computer readable program instructions. Each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Aspects of the present invention are described herein with reference to flowchart illustrations and/or block diagrams of methods, apparatus (systems), and computer-readable storage devices having instructions stored therein for carrying out functions according to embodiments of the invention. It will be understood that each block of the flowchart illustrations and/or block diagrams, and combinations of blocks in the flowchart illustrations and/or block diagrams, can be implemented by computer readable program instructions. Each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

Aspects of the present invention are described herein with reference to flowchart illustrations and/or block diagrams of methods, apparatus (systems), and computer-readable storage devices having instructions stored therein for carrying out functions according to embodiments of the invention. It will be understood that each block of the flowchart illustrations and/or block diagrams, and combinations of blocks in the flowchart illustrations and/or block diagrams, can be implemented by computer readable program instructions. Each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The various functions of the computer or other processing systems may be distributed in any manner among any number of software and/or hardware modules or units, processing or computer systems and/or circuitry, where the computer or processing systems may be disposed locally or remotely of each other and communicate via any suitable communications medium (e.g., LAN, WAN, Intranet, Internet, hardwire, modem connection, wireless, etc.). For example, the functions of the present invention embodiments may be distributed in any manner among the various end-user/client and server systems, and/or any other intermediary processing devices. The software and/or algorithms described above and illustrated in the flowcharts may be modified in any manner that accomplishes the functions described herein. In addition, the functions in the flowcharts or description may be performed in any order that accomplishes a desired operation.

In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

1. A machine-implemented method for identifying for treatment an epileptogenic zone in a brain of a person diagnosed with epilepsy, the machine-implemented method comprising:
parameterizing, by a computing device, a dynamical network model by a plurality of state transition matrices based on a plurality of neural state vectors formed from interictal data generated by monitoring each node of a plurality of nodes of the brain during each of a plurality of consecutive predefined time windows, each of the plurality of nodes corresponding to a respective area of the brain being monitored;
calculating, by the computing device for each of a plurality of state transition matrices, a corresponding node influence-to network score and a corresponding node influenced-by network score for each node of the plurality of nodes, the corresponding node influence-to network score indicating how influential the respective node is regarding the each of the plurality of nodes and the node influenced-by score indicating an amount by which the respective node is influenced by the plurality of nodes;
calculating, by the computing device for the each state transition matrix, a sink index, a source influence index for the each respective node, and a sink connectivity index for the each respective node, the sink index for the each respective node indicating how far the each respective node is from an ideal sink when one of rows and columns of a two-dimensional representation of the plurality of nodes is arranged according to a rank of the each respective node with respect to the node influence-to network score and another of the rows and the columns of the two-dimensional representation of the plurality of nodes is arranged according to a rank of the each respective node with respect to the node influenced-by network score, the source influence index for the each respective node being based on a sum of an influence of the plurality of nodes on a respective node weighted by a source index of the node, and the sink connectivity index of the each respective node being based on a sum of an influence of the plurality of nodes weighted by a sink index of the each node;
calculating, by the computing device, a score for the each respective node based on the source influence index, the sink index, and the sink connectivity index for the respective node;
determining, by the computing device, nodes of the plurality of nodes that are in the epileptogenic zone based on the calculated score for each of the plurality of nodes; and
providing an indication of the nodes determined to be in the epileptogenic zone for clinicians to plan a surgical treatment involving the epileptogenic zone.

2. The machine-implemented method of claim 1, wherein a first plurality of nodes are determined to be in the epileptogenic zone when a corresponding average score over the plurality of state transition matrices of each node of the first plurality of nodes is greater than a predefined percentage of corresponding average scores of the plurality of nodes.

3. The machine-implemented method of claim 1, further comprising:
training, by the computing device, a predictive model to estimate a probability of a successful outcome based on training data regarding each respective patient of a plurality of patients, the training data including first nodes labeled as being in a seizure onset zone, a clinically annotated epileptogenic zone including the first nodes, and second nodes not included in the clinically annotated epileptogenic zone, wherein successful outcomes are defined as a patient being seizure free after more than 12 months post-op, and failed outcomes are defined as the patient having a seizure recurrence at more than 12 months post-op; and
determining, by the computing device, a probability of success based on the trained predictive model, using an average sink index of nodes of the plurality of nodes determined to be in the epileptogenic zone, an average sink index of all nodes outside of the epileptogenic zone, an average source influence index of the nodes determined to be in the epileptogenic zone, and an average source influence index of the nodes determined to be outside of the epileptogenic zone.

4. The machine-implemented method of claim 3, wherein:
the predictive model is a logistic regression model, and
the training of the logistic regression model is based on
$$log ( \frac{{p}_{s}}{1 - {p}_{s}} ) = {β}_{0} + {β}_{1} ( {sink}_{E Z} - {sink}_{n o n E Z} ) + {β}_{2} ( s r {c}_{E Z} - s r {c}_{n o n E Z} ) + {β}_{3} ( c o n {n}_{E Z} - con {n}_{n o n E Z} ) ,$$
where pₛ is the probability of success, sink_{EZ} is the average sink index over the nodes in the clinically annotated epileptogenic zone, sink_{nonEZ} is the average sink index over the nodes outside of the clinically annotated epileptogenic zone, src_{EZ} is the average source influence index over the nodes in the clinically annotated epileptogenic zone, srC_{nonEZ} is the average source influence index over the nodes outside of the clinically annotated epileptogenic zone, conn_{EZ} is an average sink connectivity index over the nodes in the clinically annotated epileptogenic zone, conn_{nonEZ} is the average sink connectivity index over the nodes outside of the clinically annotated epileptogenic zone.

5. The machine-implemented method of claim 3, wherein when the determined probability of success is greater than a threshold value, a successful outcome is predicted.

6. The machine-implemented method of claim 1, wherein the interictal data is generated based on invasive monitoring of the brain for a time period from between 30 seconds to 60 minutes.

7. The machine-implemented method of claim 1, further comprising:
generating, by the computing device, a heat map for the plurality of nodes, the generating comprising:
for each respective state transition matrix corresponding to a respective predefined time window:
calculating, by the computing device, a respective score for the each respective node, the respective score being calculated by multiplying, based on the respective state transition matrix, a source influence index for the respective node, a sink index for the respective node, and a sink connectivity index for the respective node to produce the respective scores for the respective nodes during the respective time windows,
assigning a respective color to the each respective node in the each respective time window based on a corresponding range of values that includes the respective score for the each respective node in the corresponding time window, and
generating and presenting the heat map including one of rows and columns representing each of the respective nodes and another of the rows and columns representing respective predefined time windows arranged in chronological order, intersections of rows with columns forming cells, each of the cells representing a specific respective node during a specific respective predefined time window, each of the cells displaying the color assigned to the specific respective node for the specific respective time window represented by the each of the cells.

8. A computing device for aiding a clinician to diagnose a patient as having epilepsy, the computing device comprising:
at least one processor; and
a memory connected to the at least one processor, wherein:
the at least one processor is configured to:
parameterize a dynamical network model by a plurality of state transition matrices based on a plurality of neural state vectors formed from interictal data generated by non-invasively monitoring each node of a plurality of nodes of the brain during each of a plurality of consecutive predefined time windows, each of the plurality of nodes corresponding to a respective area of the brain being monitored;
calculate, for each of the plurality of state transition matrices, a node influence-to network score and a node influenced-by network score, respectively, for each respective node of the plurality of nodes, the node influence-to network score indicating how influential the respective node is regarding the each of the plurality of nodes, and the node influenced-by network score indicating an amount by which a respective node is influenced by the plurality of nodes;
for each respective state transition matrix corresponding to a respective predefined time window:
calculate a score for the each respective node, the respective score being calculated as a function, based on the respective state transition matrix, a source influence index for the respective node, a sink index for the respective node, and a sink connectivity index for the respective node to produce the respective score for the each respective node for the respective predefined time window, the sink index for the each respective node indicating how far the each respective node is from an ideal sink when one of rows and columns of a two-dimensional representation of the plurality of nodes is arranged according to a rank of the each respective node with respect to the node influence-to network score and another of the rows and the columns of the two-dimensional representation of the plurality of nodes is arranged according to a rank of the each respective node with respect to the node influenced-by network score, the source influence index for the each respective node being based on a sum of an influence of the plurality of nodes on a respective node weighted by a source index of each node, and the sink connectivity index of the each respective node being based on a sum of an influence of the plurality of nodes weighted by a sink index of each node;
calculate a mean score for each of the plurality of nodes based on the calculated score for each of the plurality of nodes over the each respective state transition matrix; and
normalize the mean score for the each of the plurality of nodes; and
count a number of nodes having mean scores greater than
$$\frac{1}{N} ,$$
where the N is a total number of nodes, wherein:
when the count of the number of nodes is greater than a predefined percentage of the total number of nodes, epilepsy is indicated, and
when the count of the number of nodes is less than or equal to the predefined percentage of the total number of nodes, a healthy brain is indicated.

9. The computing device of claim 8, wherein the at least one processor is further configured to:
assign a respective color to the each respective node in the each respective time window based on a corresponding range of values that includes the respective score for the each respective node in the each respective time window, and
generate and present a heat map including one of rows and columns representing each of the respective nodes and another of the rows and columns representing respective predefined time windows arranged in chronological order, intersections of rows with columns forming cells, each of the cells representing a specific respective node during a specific respective predefined time window, each of the cells displaying the color assigned to the specific respective node for the specific respective time window represented by the each of the cells.

10. The computing device of claim 8, wherein the at least one processor is further configured to:
receive the interictal data generated from a scalp electroencephalogram of the patient.

11. The computing device of claim 8, wherein the at least one processor is further configured to:
receive the interictal data generated from a non-invasive magnetoencephalogram of a brain of the patient.

12. At least one non-transitory computer-readable storage medium having computer instructions stored thereon for identifying an epileptogenic zone in a brain of a person diagnosed with epilepsy, when executed by at least one processor of a computing device, the computing device is configured to perform:
parameterizing a dynamical network model by a plurality of state transition matrices based on a plurality of neural state vectors formed from interictal data generated by invasive monitoring of each node of a plurality of nodes of the brain during each of a plurality of consecutive predefined time windows, each of the plurality of nodes corresponding to a respective probe implanted in a respective area of the brain;
calculating, based on the each respective state transition matrix, a sink index for each of the plurality of nodes, a source influence index for the each of the plurality of nodes, and a sink connectivity index for the each of the plurality of nodes, the sink index for the each respective node indicating how far the each respective node is from an ideal sink when one of rows and columns of a two-dimensional representation of the plurality of nodes is arranged according to a rank of the each respective node with respect to the influence-to score and another of the rows and the columns of the two-dimensional representation of the plurality of nodes is arranged according to a rank of the each respective node with respect to the influenced-by score, the source influence index for each respective node being based on a sum of an influence of the plurality of nodes on a respective node weighted by a source index of each node, and the sink connectivity index of the each respective node being based on a sum of an influence of the plurality of nodes on the each respective node weighted by a sink index of each node;
calculating a score for the each respective node based on an average of the source influence index, an average of the sink index, and an average of the sink connectivity index for the respective node over the plurality of state transition matrices; and
determining nodes of the plurality of nodes that are in the epileptogenic zone based on the calculated score for the each respective node of the plurality of nodes; and
providing an indication of the determined nodes in the epileptogenic zone for clinicians to plan a surgical treatment involving the epileptogenic zone.

13. The at least one non-transitory computer-readable storage medium of claim 12, wherein the calculating of the score for the each respective node further comprises:
calculating a function (e.g., the product) of the average of the sink index, the average of the source influence index, and the average of the sink connectivity index for the each respective node to produce the score for the each respective node.

14. The at least one non-transitory computer-readable storage medium of claim 12, wherein the interictal data is generated from 30 seconds to 60 minutes of the invasive monitoring.

15. The at least one non-transitory computer-readable medium of claim 12, wherein a first plurality of nodes are determined to be in the epileptogenic zone when a corresponding score of each node of the first plurality of nodes is greater than a threshold value.

16. The at least one non-transitory computer-readable medium of claim 12, wherein a first plurality of nodes are determined to be outside of the epileptogenic zone when a corresponding score of each node of the first plurality of nodes is less than a threshold value.

17. The at least one non-transitory computer-readable medium of claim 12, wherein when executed by the at least one processor of the computing device, the computing device is configured to perform:
training a predictive model to estimate a probability of a successful outcome based on training data regarding each respective patient of a plurality of patients, the training data including a first plurality of nodes labeled as being in a clinically annotated epileptogenic zone, and a second plurality of nodes indicated as being outside of the clinically annotated epileptogenic zone, wherein successful outcomes are defined as a patient being seizure free after more than 12 months post-op, and failed outcomes are defined as the patient having a seizure recurrence at more than 12 months post-op; and
determining a probability of success based on the trained predictive model, using an average sink index of nodes of the plurality of nodes determined to be in the epileptogenic zone, an average sink index of nodes of the plurality of nodes determined to be outside of the epileptogenic zone, an average source influence index of the nodes of the plurality of nodes determined to be in the epileptogenic zone, an average source influence index of the nodes of the plurality of nodes determined to be outside of the epileptogenic zone, an average sink connectivity index of the nodes of the plurality of nodes determined to be in the epileptogenic zone, and an average sink connectivity index of the nodes of the plurality of nodes determined to be outside of the epileptogenic zone.

18. The non-transitory computer-readable medium of claim 12, wherein the interictal data is generated based on the invasive monitoring of the brain for less than 60 minutes.
